(19) United States
(12) Patent Application Publication
(10) Pub. No.: US 2015/0203825 A1
(43) Pub. Date: Jul. 23, 2015

(54) Laccase of Podospora Anserina and Uses of Same

(71) Applicants: Niolas Mano, Talence (FR); Fabien Durand, Saint-Cyr-L'Ecole (FR)

(72) Inventors: Niolas Mano, Talence (FR); Fabien Durand, Saint-Cyr-L'Ecole (FR)

(21) Appl. No.: 14/399,704

(22) PCT Filed: May 21, 2013
(86) PCT No.: PCT/IB2013/054174

§ 371 Date: Nov. 7, 2014

(30) Foreign Application Priority Data
May 21, 2012 (FR) 1254615

(51) Int. Cl. C12N 9/02; H01M 8/16

(52) U.S. Cl. CPC C12N 9/0061; H01M 8/16; C12Y 110/03002

(57) ABSTRACT

The present invention relates to a novel laccase of Podospora anserina, to the method for preparing same and to the use thereof, particularly for delignifying paper, as a bleaching, depolluting and deodorising agent, or even for lowering oxygen content.

The present invention relates to a novel laccase isolated from Podospora anserina, to the method for preparing same and also to the use thereof in particular for delignifying paper, as a bleach, depolluting and deodorizing agent or else for reducing oxygen.

Laccases are copper-containing enzymes which oxidize polyphenols using oxygen as final electron acceptor. They are present in plants, in a very large number of fungi (lignin degradation) and also in some bacteria.

The structure of laccases is such that their active site consists of 4 copper atoms, one of type 1 (T1), which is isolated and is responsible for phenol oxidation, and a cluster of 3 copper atoms (one of type 2, T2, and two of type 3, T3) responsible for O₂ activation.

The mechanism of action of laccases and in particular the role of the metallic center remain poorly understood; a two-step mechanism is accepted:
1. the T1 copper drags an electron from the substrate;
2. the electron is transferred to the T2/T3 center over a distance of approximately 12.5 Å. After complete reduction of the trinuclear center, the reduction of molecular oxygen takes place.

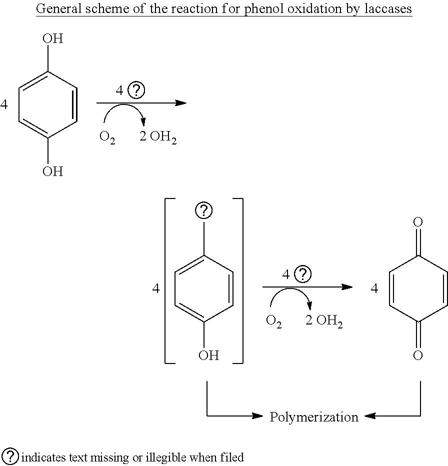

The inventors have now identified a novel laccase produced by Podospora anserina which exhibits characteristics that are more advantageous than the commercially available laccases, in particular that of Trametes versicolor.

According to a first subject, the invention relates to the laccase purified and isolated from Podospora anserina, of SEQ ID No. 1; this enzyme corresponds to a protein predicted by sequencing of the Podospora anserina genome (translated from the gene having accession number B2ANK8 in the UniProt database; this complete gene has the sequence SEQ ID No. 2) with the exception of the 30 amino acids positioned at the N-terminal end of the protein; in particular, the laccase purified (purity>95%) according to the invention has a percentage identity of at least 90%, and, in increasing order of preference, at least 95%, 97%, 98% and 99% identity, relative to the Podospora anserina laccase of SEQ ID No. 1; according to one particular variant, it comprises a total or partial truncation of 30 amino acids positioned at the N-terminal end; it catalyzes the reaction for oxidation of phenol nuclei and is bonded to four copper atoms; the nucleic acid sequence SEQ ID No. 4 (corresponding to SEQ ID No. 2 with the exclusion of the fragment encoding the 30 amino acids positioned at the N-terminal end) encodes this laccase of SEQ ID No. 1.

The laccase of SEQ ID No. 3 is also a subject of the present invention, the laccase of SEQ ID No. 3 differs from the laccase of SEQ ID No. 1 in that it comprises an additional amino acid, a serine, in the N-terminal position; this laccase is obtained by expressing, in the yeast Pichia pastoris, the nucleic acid molecule of SEQ ID No. 4 cloned into the pFD56 expression vector, the construction of which is such that it enables the expression of a protein bearing an additional serine in the N-terminal position.

The laccase of SEQ ID No. 5, which corresponds to the polypeptide encoded by SEQ ID No. 2 and which comprises the 30 amino acids of the N-terminal end, is also a subject of the present invention.

Generally, the inventors have noted that the modifications of sequences introduced at the N-terminal end, whether this involves addition of amino acids or substitution, deletion or insertion within the N-terminal end, within the limit of maintaining an identity of at least 90% with SEQ ID No. 1, do not affect the enzymatic properties of the laccase according to the invention. Thus, one variant of the invention relates to a laccase of SEQ ID No. 1 comprising at least one modification (substitution, deletion or insertion) within the sequence of the 30 amino acids positioned at the N-terminal end, the other amino acids not located in this sequence preferably being unchanged and the identity of this enzyme being at least 90%, preferably 95%, 97%, 98% and 99%, with SEQ ID No. 1.

The identity of a sequence relative to the sequence of the Podospora anserina laccase (SEQ ID No. 1) as reference sequence is assessed according to the percentage of amino acid residues which are identical, when the two sequences are aligned, so as to obtain the maximum correspondence between them.

The percentage identity can be calculated by those skilled in the art using a sequence comparison computer program such as, for example, that of the BLAST series (Altschul et al., NAR, 25, 3389-3402). The BLAST programs are used on the comparison window consisting of all of SEQ ID No. 1 indicated as reference sequence.

A peptide having an amino acid sequence which has at least X % identity with a reference sequence is defined, in the present invention, as a peptide of which the sequence can include up to 100-X modifications for 100 amino acids of the reference sequence, while at the same time retaining the functional properties of said reference peptide. For the purposes of the present invention, the term “modification” includes consecutive or dispersed deletions, substitutions or insertions of amino acids in the reference sequence.

A protein sequence SEQ ID No. 5 predicted from the systematic sequencing of the Podospora anserina genome is described in the UniProt database (accession number B2ANK8); it should be emphasized that the information presented in the UniProt database is predictive and putative, it does not result from the experimental isolation and characterization of Podospora anserina proteins. In addition, the UniProt database does not indicate any precise enzyme activity for this predicted protein.

The novel laccase according to the invention exhibits improved properties compared with the commercially available laccases derived from Trametes versicolor, Rhus vernicifera, Agaricus bisporus or else Pleurotus ostreatus.

The present invention also relates to a nucleic acid molecule encoding the laccase according to the invention; preferably, it is a nucleic acid molecule of sequence chosen from SEQ ID No. 2 or, preferentially, it is SEQ ID No. 4 encoding the Podospora anserina laccase cleaved at the level of the first 30 amino acids positioned at the N-terminal end of the protein.

The nucleic acid molecule encoding the laccase according to the invention can be cloned into an expression vector such as a plasmid, then an appropriate host, such as a bacterium, a yeast or else a cell culture, can be transformed therewith.

The term “expression vector” is intended to mean a vector which has a region enabling the insertion of a coding nucleotide sequence between the signals essential for its expression, in particular a (constitutive or inducible) promoter, a ribosome-binding site, a transcription termination signal and, optionally, a selectable marker such as a gene for resistance to an antibiotic.

The present invention also relates to an expression vector comprising said nucleic acid molecule and to a host cell transformed with said expression vector and expressing a laccase according to the invention.

The introduction of the expression vector into the host cell can be carried out by any method known to those skilled in the art, in particular by modification of the membrane permeability of the host cell, for example in the presence of calcium ions, or by electroporation.

After culturing of the host cells transformed so as to express the laccase according to the invention, said cells can be recovered by centrifugation, and lysed in order to release the enzymes, including said laccase according to the invention.

According to one preferred variant of the invention, the laccase according to the invention is produced by the yeast Pichia pastoris.

In order to enable the overproduction and the secretion of the laccase into the Pichia pastoris yeast culture medium, the nucleic acid molecule SEQ ID No. 4, encoding the laccase of sequence SEQ ID No. 3 when it is cloned into the pFD56 vector as described hereinafter, is introduced into the yeast genome, at the level of the AOX1 gene, by homologous recombination. For this, the pFD56 plasmid, once linearized by digestion with the pmeI enzyme, is introduced into the yeast by electroporation and the positive clones are selected on YPD medium+agar containing zeocin at 100 μg/ml. A preculture of 200 ml of YPD medium supplemented with zeocin (100 μg/ml) is inoculated using a clone isolated on a Petri dish. After shaking overnight at 220 rpm and at 30° C., this preculture is then centrifuged for 10 minutes at 4000 rpm and the pellet is taken up in 200 ml of sterile water in order to remove any presence of glucose. After a second centrifugation, a 21 culture in MMH medium containing 1 mM of CuSo₄ in a 51 Erlenmeyer flask is then inoculated with this pellet. The yeasts are incubated at 25° C. with shaking (220 rpm) for 2 hours before 0.5% of methanol is added in order to initiate the induction. This induction step will be repeated for 5 days in order to obtain the maximum amount of enzymes.

The following material can, without being limiting in nature, be used to carry out this method:
vector for expression in Pichia pastoris (pFD56): pPICZα plasmid containing the DNA sequence encoding the Podospora anserina laccase in frame with the α-factor secretion factor of Saccharomyces cerevisiae and containing the methanol-inducible AOX1 promoter;
Pichia pastoris yeast strain GS115 used for the production of the laccase according to the invention after integration of the cassette derived from the pFD56 vector containing the AOX1 promoter, the α-factor signal peptide and the DNA sequence encoding the Podospora anserina laccase;
culture media:

### YPD Rich Medium (for Yeast):

1% yeast extract
2% bactopeptone
2% glucose
pH not adjusted, autoclaved for 20 min at 120° C.

### MMH Minimum Medium (for Yeast):

1.34% yeast nitrogen base
1% casamino acid
0.4% histidine
4×10⁻⁵% biotin
pH not adjusted, autoclaved for 20 min at 120° C.

### LB Rich Medium (for Bacteria):

Tryptone 10 g/l
Yeast extract 5 g/l
NaCl 5 g/1
Distilled H₂O qs 11
pH not adjusted, autoclaved for 20 min at 120° C.

According to another variant, Escherichia coli can be chosen as host microorganism; the plasmids which can then be used are in particular the plasmids pBluescript, pUC18, pET, pGEX, pGS, μMAL-c2 or the like.

According to this variant of preparation of the laccase according to the invention, the laccase is advantageously expressed by an E. coli bacterium transformed with a pET21a expression vector encoding an enzyme placed side by side with a 6HIS tag in the C-terminal position.

This method is rapid and simple; indeed, the induction of the expression of the Podospora anserina laccase in the E. coli bacterium is carried out in 4 to 24 hours.

In addition, the 6HIS tag enables the purification of the Podospora anserina laccase by affinity chromatography on a nickel resin in a single step in order to obtain a pure enzyme.

To carry out this preparation method, those skilled in the art choose the host cell according to the expression vector used.

Preferably, when the pET21a expression vector is used, a host cell expressing the T7 RNA polymerase is chosen, such as the E. coli strains BL₂₁, DE3, BL₂₁-SI, BL₂₁ pLys, Novablue(DE3) or BL₂₁ Star.

The present invention also relates to a method for preparing a laccase according to the invention, comprising the steps of:
a) preparing host cells, expressing the laccase according to the invention;
b) culturing the host cells prepared in step a);
c) recovering the culture medium and removing the host cells, for example by centrifugation;
d) treating the culture medium obtained in step c) by hydrophobic interaction chromatography;
e) recovering said purified laccase.

According to one preferred embodiment, the method according to the invention is such that:
the Pichia pastoris yeast strain used is the GS115 strain;
the vector for expression in Pichia pastoris (pFD56) is the pPICZα plasmid containing the DNA sequence encoding the Podospora anserina laccase in frame with the α-factor secretion factor of Saccharomyces cerevisiae and containing the methanol-inducible AOX1 promoter;
the culturing carried out in step b) comprises at least one step of culturing in liquid phase, with shaking, at a temperature of between 18 and 37° C., preferably 25° C., during which the expression of the laccase is induced by adding methanol, it being possible for the induction by adding methanol to be optionally repeated.

When the method is carried out according to these preferred conditions, it enables the production of the laccase with a short induction time, of about 3 to 7 days; the purification of the laccase is carried out in a single hydrophobic interaction chromatography step and the laccase thus produced indeed comprises the four copper atoms required for its activity.

When the laccase according to the invention is produced by a strain such as Escherichia coli, then the method for preparing a laccase according to the invention comprises the steps of:
a) preparing host cells, expressing the laccase according to the invention;
b) culturing the host cells prepared in step a);
c) lysing the host cells;
d) treating the lysate obtained in step c) by affinity chromatography;
e) recovering said purified laccase.

It is also possible to produce a laccase in the presence of denaturing agents such as urea, guanidinium chloride, SDS, triton, etc.; the laccase thus produced will then be devoid of copper and may be activated by adding copper atoms.

The Applicant has demonstrated that the laccase according to the invention exhibits electrochemical properties which are better than the commercially available laccases (see point 10 of the experimental section), in particular that of Trametes versicolor.

Thus, by virtue of their advantageous enzymatic properties, the laccases according to the invention are of particular interest in the following applications:

Use in the Paper Industry

The most customary applications of laccases relate to the delignifying and/or bleaching of paper pulp.

In the industrial preparation of paper, the separation and the degradation of the lignin in wood pulp are conventionally obtained using chlorine and/or chemical oxidants; these conventional methods have the drawback of being polluting. The laccases according to the invention offer an advantageous alternative for carrying out the delignifying but also the bleaching of the paper pulp without chlorine.

Use as a Bleaching Agent

The paper-bleaching capacity that laccases have can be applied in other fields: the laccases according to the invention can also be used to remove ink from paper and/or to bleach it, in particular with a view to recycling it; they can also be used for treating fabrics, in particular bleaching cotton, or for producing the bleached indigo color of jeans.

Use as a Depolluting Agent

Another very frequent application of laccases relates to their use as a depolluting agent; this enzyme can in fact degrade a broad spectrum of undesirable environmental contaminants including phenols (optionally chlorinated phenolic pollutant) and plastics. An advantageous use of the laccases according to the invention thus relates to the depollution of phenol-based products.

Use as a Deodorizing Agent and in Detergent Compositions

By virtue of their capacity to degrade pollutants, laccases also make it possible to deodorize materials such as fabrics; they are thus of use in detergent compositions for washing laundry or doing the dishes.

Use in the Food Industry

Laccases are used in the food industry as a food product preservative, in particular as an additive in order to eliminate oxidizing reagents (deoxygenation), for example for the stabilization of fruit juices and of wine.

They are also used as a food product taste enhancer; for example, they are involved in the method for preparing cocoa for enhancing its taste (soaking in a laccase solution before drying and roasting).

The laccases according to the invention also have the advantage of enabling the crosslinking of whey proteins to give oligomers or polymers and thus leading to the formation of gels (Faergemand et al. 1998 J. Agric. Food Chem. 46, 1326-1333; Mattinen et al. 2005 FEBS Journal 272, 3640-3650).

Use for Carrying Out Organic Syntheses

Laccases are involved in the process for producing ethanol from recycled raw material.

Use in the Pharmaceutical Field

Here again, laccases are used for the synthesis of anesthetic, anti-inflammatory or antibiotic compounds or else of iodine.

Use in the Cosmetics Field

Numerous developments are made in the cosmetics field; these are in particular use as reagents in oxidation dyeing compositions where the laccases enable the preparation of a less irritant product. They also enable the preparation of a skin lightening product.

Finally, they can be used in deodorants or hygiene products such as soap and toothpaste.

Use for the Fabrication of Electrodes and of Biofuel Cells

The laccase according to the invention is of particular interest for the fabrication of electrodes on which the enzyme is immobilized.

Thus, the present invention also relates to laccase-containing electrodes comprising a conductive material, such as a conductive metal, in particular platinum, copper, silver, aluminum, gold or steel, or carbon, for instance vitreous carbon, carbon fibers, fibers of carbon nanotubes, or else those made of diamond, etc; said conductive material is covered with a deposit comprising at least one laccase according to the invention, it being possible for said deposit to also comprise a redox polymer in order to improve the electrical conduction between the enzyme and the electrode and also the stability of the system.

The redox polymer may, for example, be chosen from ferrocene-, osmium- and ruthenium-based polymers and conductive polymers such as, for example, polypyrrole and polyaniline.

The methods for immobilizing the laccase on said conductive material can be chosen from the conventional methods at the disposal of those skilled in the art, which comprise in particular the inclusion of the laccase in a polymer matrix, the adsorption of the laccase at the surface of the polymer membrane, attachment by covalent bonding, electrodeposition (Gao et al., Chem. Int. ED. 2002, 41, No. 5, 810-813) or else the technique described in United States patent application US 2009/0053582.

According to one implementation variant, the laccase-containing electrode on which the laccase is immobilized is also covered with a membrane which prevents the detachment of said enzyme from the electrode. According to the applications envisioned, said membrane can consist of nafion, of cellulose or of any material which is biocompatible, i.e. which is compatible with a physiological environment.

The present invention thus also relates to an oxygen, phenolic-compound or aromatic-amine biosensor, consisting of a laccase-containing electrode according to the invention. Generally, a biosensor consists of an electrode on which is immobilized a bioreceptor capable of recognizing a biological target; the binding of the biological target to the bioreceptor results in physicochemical modifications of the membrane and the production of an electrical signal by an electrochemical transducer (amperometric, potentiometric, conductometric, etc.) attached to the electrode. In the present case, the bioreceptor is a laccase according to the invention and the biological target is a compound chosen from oxygen, phenolic compounds or aromatic amines.

The present invention also relates to an oxygen sensor consisting of an electrode according to the invention.

The laccase-containing electrode according to the invention can also be advantageously used as a cathode in an enzymatic biofuel cell; FIG. 1A represents schematically the principle of operation of an enzymatic biofuel cell. The enzymatic biofuel cells according to the invention are devices comprising a laccase-containing electrode (lacca) as cathode and an anode where a substrate oxidation reaction takes place (catalyzed by the “enzyme X”); by way of illustration, the substrate may be glucose and the “enzyme X” may be glucose oxidase, such a fuel cell is of particular interest when the biofuel cell is implanted in an individual for a medical application; the substrate may also be chosen, for example, from nitrites, nitrates, sulfides, urates, ascorbates, glutamates, pyruvates, lactates, cellulose, etc.; if an application in depollution is envisioned, the choice of the enzyme will then be made according to the substrate to be degraded; by way of example, the following enzymes can be used, the type of substrate that they can degrade being mentioned between parentheses: glucose oxidase (glucose or any of the sugars which are oxidized by this enzyme), lactate oxidase (lactate), pyruvate oxidase (pyruvate), alcohol oxidase (alcohol), cholesterol oxidase (cholesterol), glutamate oxidase (glutamate), pyranose oxidase (pyranose), choline oxidase (choline), cellobiose dehydrogenase (cellobiose), glucose dehydrogenase (glucose or any of the sugars which are oxidized by this enzyme), pyranose dehydrogenase (pyranose), fructose dehydrogenase (fructose), aldehyde oxidase (aldehyde), gluconolactam oxidase (gluconolactam), alcohol dehydrogenase (alcohol), ascorbate oxidase (oxygen or ascorbate) or else sulfur dioxygenase (sulfur). The concomitant oxidation and reduction process at the electrodes of the biofuel cell produces an electric current.

FIG. 1B shows more specifically a glucose enzymatic biofuel cell; such an enzymatic biofuel cell consists of two electrodes modified by the immobilization of enzymes. A glucose oxidase (GOx) is attached to the anode (1) by means of a conductive polymer “I” and a laccase (LAC) is attached to the cathode (2) by means of a conductive polymer “II”. When operating, at the anode, the electrons are transferred from the glucose present in the physiological fluid to the GOx, then from the GOx to the conductive polymer “I” and from the conductive polymer “I” to the anode. At the cathode, the electrons are transferred from the cathode to the conductive polymer “II”, then to the laccase and, finally, from the laccase to the oxygen present in the physiological fluid.

It should be noted that a biofuel cell can also optionally operate by modifying the electrodes with their respective enzymes and by adding soluble mediators, such as ferrocenemethanol for the anode and potassium ferricyanide for the cathode, and by adding, where appropriate, a membrane separating the anode and the cathode.

Such a biofuel cell can be used as a miniaturized energy source and can be implanted in a living organism.

In addition to the above arrangements, the invention also comprises other arrangements which will emerge from the description which will follow, which refer to examples of implementation of the present invention, and also to the appended figures in which:

### FIGURES

FIG. 1A represents schematically the principle of operation of an enzymatic biofuel cell; FIG. 1B represents a glucose enzymatic biofuel cell.

FIG. 2 represents the plasmid map of the pFD56 vector.

FIG. 3 is a graph representing the catalytic activity of the laccase of SEQ ID No. 3 according to the invention as a function of the ABTS concentration at 37° C.

FIG. 4 is a graph representing the catalytic activity of the laccase of SEQ ID No. 3 according to the invention as a function of the SGZ concentration at 37° C.

FIG. 5 is a curve representing the relative activity of the laccase of SEQ ID No. 3 according to the invention as a function of the pH, on the oxidation of ABTS.

FIG. 6 shows the stability of the laccase of SEQ ID No. 3 according to the invention at various pH values at 4° C.

FIG. 7 is a graph representing the relative activity of the laccase of SEQ ID No. 3 according to the invention as a function of the temperature, on the oxidation of ABTS.

FIG. 8 is a graph representing the stability of the laccase of SEQ ID No. 3 according to the invention as a function of time, at 37° C. and 60° C., on the oxidation of ABTS.

FIG. 9 represents the effect of NaCl on the activity of the laccase of SEQ ID No. 3 according to the invention at pH 7.

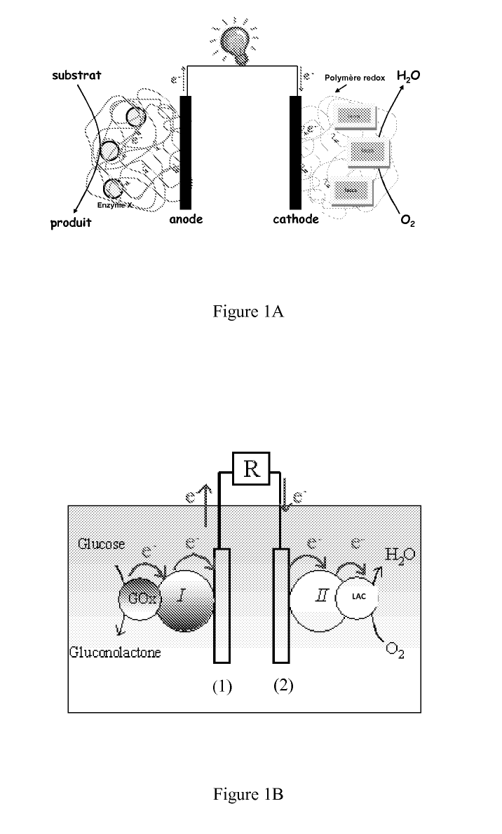

### EXAMPLE

### I. Preparation of the Laccase Derived from the Fungus Podospora anserina

### Materials

1. Escherichia coli Bacterial Strain

DH₅α: supE44, ΔlacU169, (θ80 lacZDM15), hsdR17, recA1, endA1, gyrA96, thi-1, relA1 (Hanahan, 1983). This strain is used for plasmid amplification during the protein expression vector construction steps.

2. Vector

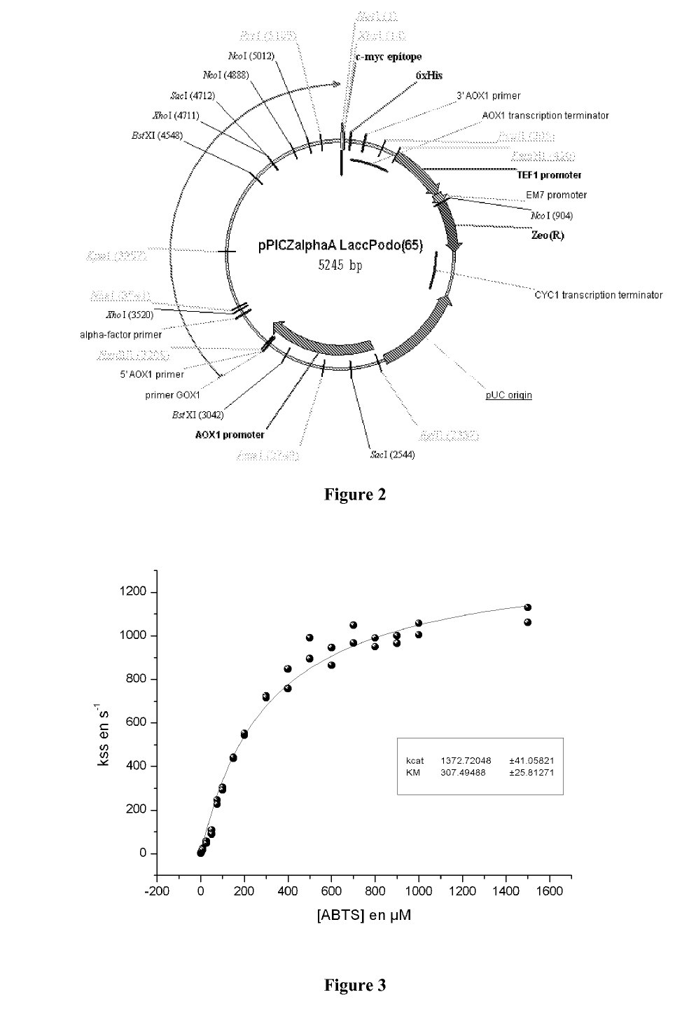

pFD56: pPICZα plasmid containing the DNA sequence encoding the laccase of SEQ ID No. 3 according to the invention from Podospora anserina in frame with the α-factor secretion factor of Saccharomyces cerevisiae and containing the methanol-inducible AOX1 promoter; the plasmid is represented in FIG. 2.

3. Pichia pastoris Yeast Strain

GS115: Pichia pastoris yeast strain used to produce the laccase after integration of the cassette derived from the pFD56 vector containing the AOX1 promoter, the α-factor signal peptide and the DNA sequence encoding the laccase of SEQ ID No. 3 according to the invention from Podospora anserina.

4. Culture Medium

YPD Rich Medium (for Yeast):
1% yeast extract
2% bactopeptone
2% glucose
pH not adjusted, autoclaved for 20 min at 120° C.

MMH Minimum Medium (for Yeast):
1.34% yeast nitrogen base
1% casamino acid
0.4% histidine
4×10⁻⁵% biotin
pH not adjusted, autoclaved for 20 min at 120° C.

LB Rich Medium (for Bacteria):
Tryptone 10 g/l
Yeast extract 5 g/l
NaCl 5 g/l
Distilled H₂O qs 1 l
pH not adjusted, autoclaved for 20 min at 120° C.

### Genetic Engineering Techniques

5. Transformation of Supercompetent Bacteria

The supercompetent DH₅, bacteria are prepared using the Inoue method (Sambrook and Russell).

6. Transformation of the Pichia pastoris Yeast

The DNA is introduced into the Pichia pastoris GS115 yeast by electroporation on an Eppendorf Eporator (Eppendorf, France).

7. DNA Preparation

A plasmid DNA purification kit (Qiagen) is used for the DNA preparations in small and large amount.

8. Double-Stranded DNA Sequencing

The double-stranded DNA is sequenced by the company Millegen (Toulouse, France).

9. Construction of the Laccase Expression Vector

The gene of sequence SEQ ID No. 4 corresponding to the sequence encoding the Podospora anserina laccase (accession B2ANK8) from which the part encoding the first 30 amino acids of the N-terminal end has been truncated, was synthesized by the company Genecust Europe (Luxembourg). The NheI and NotI restriction sites were respectively added in the 3′ position and 5′ position of the sequence in order to facilitate cloning. The pPICZα plasmid and also the synthesized gene were then treated with the two restriction enzymes NheI and NotI and the digestion products were purified on a gel with the “nucleospin” kit. The laccase gene is then ligated into the plasmid by coincubation with T4 DNA ligase at 37° C. overnight. The newly formed plasmids (pFD56) are then selected and amplified by transformation of DH5a bacteria on a dish containing zeocin at 25 μg/ml.

10. Integration of the Sequencing Encoding the Laccase into the Pichia pastoris Genome

In order to enable the overproduction and secretion of the enzyme into the culture medium of the Pichia pastoris yeast, the corresponding gene is introduced by homologous recombination at the level of the AOX1 gene. For this, the pFD56 plasmid, once linearized by digestion with the pmeI enzyme, is introduced into the yeast by electroporation and the positive clones are selected on YPD medium+agar containing zeocin at 100 μg/ml.

The sequence of the resulting plasmid is SEQ ID No. 6.

Production, Purification and Characterization of the Laccase Enzyme Derived from Podospora anserina

### Laccase Production

The laccase enzyme of SEQ ID No. 3 is produced by the Pichia pastoris yeast via methanol induction. To do this, a 200 ml preculture of YPD medium supplemented with zeocin (100 μg/ml) is inoculated with the GS115 strain having integrated the cassette contained on the pFD56 plasmid. After shaking overnight at 220 rpm and at 30° C., this preculture is then centrifuged for 10 min at 400 rpm and the pellet is resuspended in 200 ml of sterile water in order to remove any presence of glucose. After a second centrifugation, a 21 culture in MMH medium containing 2 mM of CuSO₄ in a 51 Erlenmeyer flask is then inoculated with this pellet. The yeasts are incubated at 25° C. with shaking (220 rpm) for 2 hours before 0.5% of methanol is added in order to initiate the induction. This induction step will be repeated for 5 days in order to obtain the maximum amount of enzymes. In order to recover the secreted proteins, the 21 culture is centrifuged and the supernatant containing the enzyme of interest is concentrated on a stirring cell with a YM10 membrane having a cut-threshold of 10 kDa, so as to achieve a final volume of 4-5 ml.

### Laccase Purification by Hydrophobic Interaction Chromatography

Once concentrated, 1.7 M of ammonium sulfate is added to the 4-5 ml of the culture supernatant before being filtered on a 0.22 μm filter so as to be injected onto a hydrophobic interaction chromatography column, a 60 ml PhenylHP (GE Healthcare®), coupled to the AKTA purifier system (GE Healthcare®), equilibrated in a 50 mM potassium phosphate, 1.7 M (NH₄)₂SO₄ buffer, pH 6. The elution is carried out with a gradient from 0% to 100% of a 50 mM potassium phosphate buffer, pH 6, at a flow rate of 2.5 ml/min. The fractions containing the laccase protein are identified using an ABTS activity test and are combined, concentrated and stored in a 50 mM potassium phosphate buffer, pH 6, by centrifugation on an Amicon YM10 membrane. At this stage, the protein is pure and can be stored at −20° C. in soluble form.

By comparing with other commercially available laccases, a real advantage to using this protein with respect to the purification protocol is apparent. This is because a single purification step is necessary in order to obtain a pure enzyme, as opposed to the successions of chromatography (size exclusion, anion or cation exchange, hydrophobic, etc.) used for the other enzymes [1].

### II. Characterization of the Enzyme

### 1. Measurement of Concentration

The enzyme concentration of a solution is calculated from a BSA range according to the Bradford technique [2].

### 2. Enzymatic Test

The enzymatic tests are carried out using a Varian spectrophotometer in a 0.1 M citrate/phosphate buffer at 37° C. in a volume of 3 ml by monitoring the oxidation of various substrates at a given wavelength as a function of time. The specific activity of the enzyme is expressed in μmol of substrates oxidized per minute and per mg of protein. The substrates used in this study are: ABTS (ε₄₂₀ₙₘ=36 mM⁻¹ cm⁻¹) and syringaldazine, SGZ (ε₅₃₀ₙₘ=64 mM⁻¹ cm⁻¹).

Study of the Enzymatic Properties of the Podospora anserina Laccase
Determination of the Kinetic (k_{cat}) and Michaelis (K_{M}) Constants in the Stationary Phase

### 3. ABTS

The experiments are carried out at 37° C. on a Varian spectrophotometer in a 0.1 M citrate/phosphate buffer, pH 3.4. The ABTS concentration varies in the test from 0 to 1.5 mM. The test is initiated by adding enzyme. The experimental points are analyzed by nonlinear regression according to the Michaelis-Menten model using the Sigma-plot 6.0 software according to the equation below:

### Michaelis-Menten Model

kₛₛ=k_{cat}*[S]/(K_{M}+[S])

Results:

k_{cat}=1372 s⁻¹, Kₘ=307 μM.

FIG. 3 represents the catalytic activity of the laccase of SEQ ID No. 3 as a function of the ABTS concentration at 37° C.

### 4. Syringaldazine (SGZ)

The experiments are carried out at 37° C. on a Varian spectrophotometer in a citrate-phosphate 50 mM buffer, pH 7. The concentration of SGZ, diluted in methanol, varies in the test from 0 to 50 μM. The test, initiated by the addition of enzyme, consists in monitoring the oxidation of the SGZ at 530 nm by colorimetric change (ε₅₃₀ₙₘ=64 mM⁻¹ cm⁻¹).

Results:

k_{cat}=1.3 s⁻¹, K_{M}=10.9 μM.

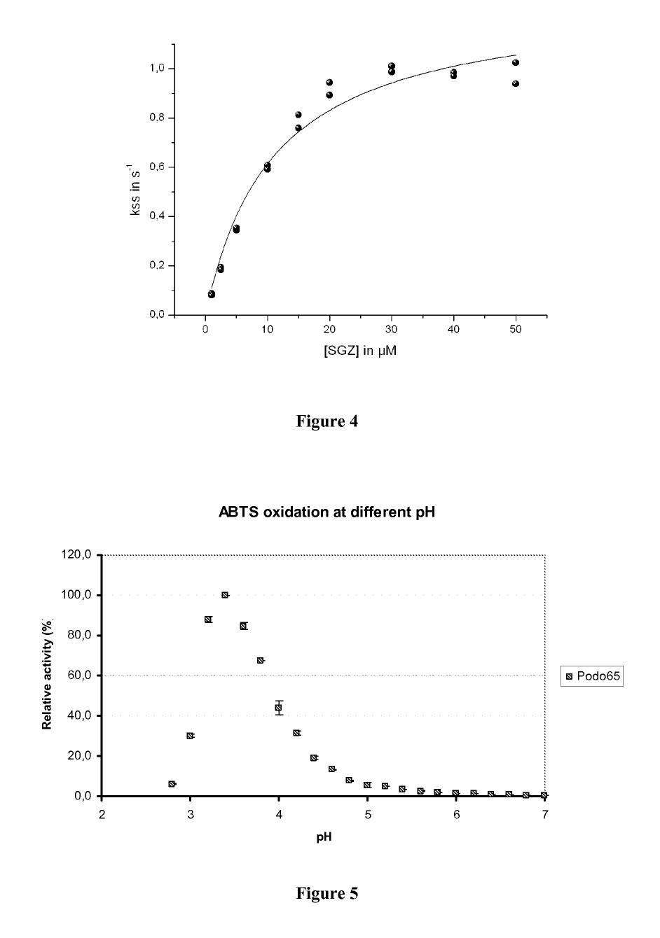

These results are presented in FIG. 4.

### 5. Activity as a Function of pH

The study of the variation of the rate constant of the reaction as a function of pH is carried out on a pH range of from 3 to 7 in a 0.1 M citrate/phosphate buffer using 1 mM ABTS as substrate. The experiments were carried out at 37° C. using a Varian spectrophotometer. The activity is monitored via the oxidation of the ABTS resulting in a colorimetric change measured at 420 nm. The test is initiated by adding the enzyme.

FIG. 5 represents the relative activity of the laccase of SEQ ID No. 3 as a function of pH, on the oxidation of ABTS.

### 6. Stability as a Function of pH

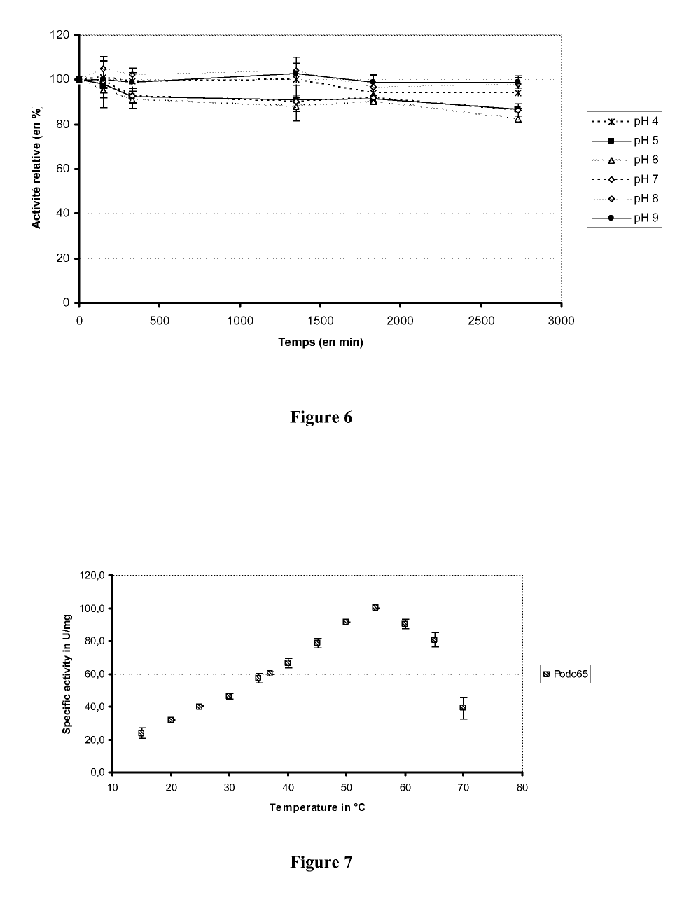

The enzyme, at a concentration of 0.15 mg/ml, is preincubated in a 50 mM phosphate-citrate buffer (or 50 mM Tris-H₂SO₄ for pH 8 and 9) at a given pH at 4° C. At regular times, 5 μl samples are taken and the residual activity of the enzyme incubated at the various pH values is determined using a Varian spectrophotometer at 440 nm in a 0.1 M citrate/phosphate buffer, pH 3.4, at 37° C., in the presence of 150 μM of ABTS. The test is initiated by adding the enzyme. The results obtained are represented in FIG. 6.

### 7. Activity as a Function of Temperature

The study of the variation in the rate constant of the reaction as a function of temperature is carried out in a 0.1 M citrate/phosphate buffer, pH 4, in the presence of 0.5 mM of ABTS. The temperature varies from 15 to 80° C. The activity is monitored on a temperature-regulated Varian CARY UV Biomelt spectrophotometer. The test is initiated by adding the enzyme. The results obtained are represented in FIG. 7.

### 8. Stability of the Enzyme as a Function of Temperature

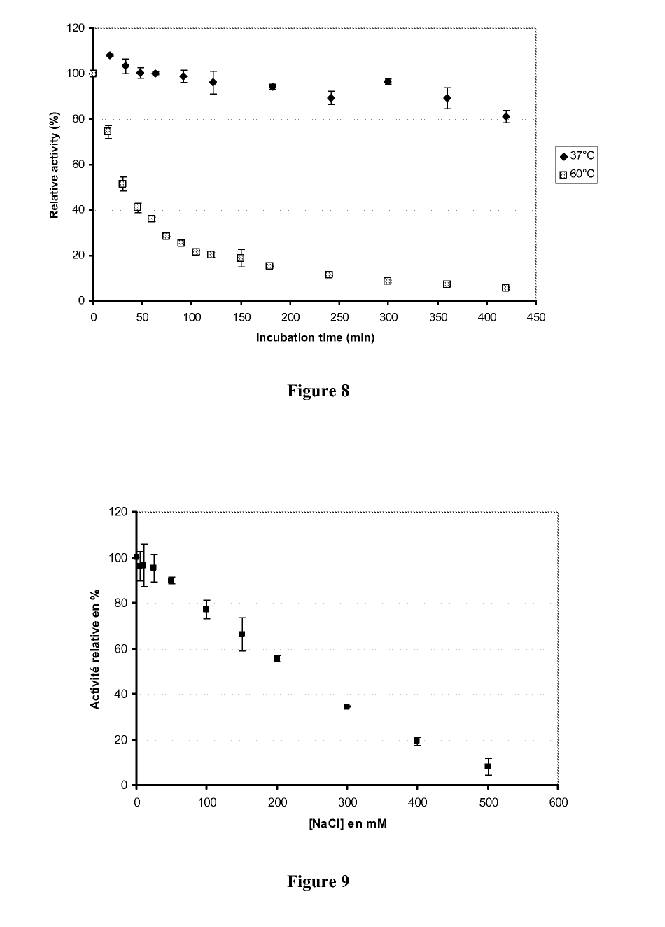

The enzyme is preincubated at a concentration of 0.15 mg/ml in a dry bath at 60° C. and at 37° C. in a 50 mM potassium phosphate buffer, pH 6. At regular times, 5 μl samples are taken and the residual activity of the enzyme incubated at these temperatures is determined using a Varian spectrophotometer at 440 nm in a 0.1 M citrate/-phosphate buffer, pH 3.4, at 37° C., in the presence of 150 μM of ABTS. The test is initiated by adding the enzyme. The results obtained are represented in FIG. 8.

### 9. Study of the Enzymatic Activity in the Presence of NaCl

In order to verify the influence of sodium chloride (present in a physiological medium) on the activity of the laccase, increasing concentrations of NaCl were added to the activity test consisting in monitoring the oxidation, at 530 nm, of the SGZ at 37° C. in a 50 mM phosphate-citrate buffer, pH 7.

As shown in FIG. 9 (effect of NaCl on the activity of the laccase at pH 7), in the presence of 150 mM of NaCl, which is a concentration corresponding to physiological conditions, the enzyme still exhibits 70% oxidation activity.

### 10. Electrochemical Measurements

In order to demonstrate the advantage of this novel enzyme and by way of comparative example, two enzymatic electrodes were prepared, comprising either the laccase of SEQ ID No. 3 according to the present invention, or the laccase of Trametes versicolor (the laccase commonly used in electrochemistry) and a redox polymer. At pH 7 and in the presence of Cl, the catalytic current for the reduction of O₂ is 40% greater for the electrode modified with the novel enzyme. In addition, the stability over time is 200% greater.

### LITERATURE REFERENCES

1. Colao, M. C., et al., Heterologous expression of lcc1 gene from Trametes trogii in Pichia pastoris and characterization of the recombinant enzyme. Microb Cell Fact, 2006. 5: p, 31.
2. Bradford, M. M., A rapid and sensitive method for the quantitation of microgram quantities of protein utilizing the principle of protein-dye binding. Anal Biochem, 1976. 72: p. 248-54.
3. Kataoka, K., et al., High-level expression of Myrothecium verrucaria bilirubin oxidase in Pichia pastoris, and its facile purification and characterization. Protein Expr Purif, 2005. 41(1): p. 77-83.
4. Sakasegawa, S., et al., Bilirubin oxidase activity of Bacillus subtilis CotA. Appl Environ Microbiol, 2006. 72(1): p. 972-5.
5. Felsenfeld, G., The determination of cuprous ion in copper proteins. Arch Biochem Biophys, 1960. 87: p. 247-51.

1. A purified laccase, characterized in that it has a percentage identity of at least 90% relative to the Podospora anserina laccase of SEQ ID No. 1, in that it catalyzes the reaction for oxidation of phenol nuclei and in that it is bonded to four copper atoms.

2. The purified laccase as claimed in claim 1, characterized in that it is chosen from the laccases of sequence SEQ ID Nos. 1, 3 and 5.

8-22. (canceled)

23. A nucleic acid molecule, characterized in that it encodes a laccase as claimed in claim 1.

24. A nucleic acid molecule, characterized in that it encodes a laccase as claimed in claim 2.

25. The nucleic acid molecule as claimed in claim 23, of sequence chosen from SEQ ID Nos. 2 and 4.

26. The nucleic acid molecule as claimed in claim 24, of sequence chosen from SEQ ID Nos. 2 and 4.

27. An expression vector, characterized in that it comprises a nucleic acid molecule as claimed in claim 23.

28. An expression vector, characterized in that it comprises a nucleic acid molecule as claimed in claim 24.

29. A host cell expressing a laccase that has a percentage identity of at least 90% relative to the Podospora anserina laccase of SEQ ID No. 1, in that it catalyzes the reaction for oxidation of phenol nuclei and in that it is bonded to four copper atoms, wherein the host cell is characterized in that it is transformed with an expression vector as claimed in claim 27.

30. A host cell expressing a laccase that has a percentage identity of at least 90% relative to the Podospora anserina laccase of SEQ ID No. 1, in that it catalyzes the reaction for oxidation of phenol nuclei and in that it is bonded to four copper atoms, wherein the host cell is characterized in that it is transformed with an expression vector as claimed in claim 28.

31. A method for preparing a laccase, comprising the steps of:
a) preparing host cells, as claimed in claim 29;
b) culturing host cells prepared in step a);
c) recovering the culture medium and removing said host cells in the case of secreted proteins, or lysing the host cells;
d) treating the medium or lysate obtained in step c) by hydrophobic interaction chromatography;
e) recovering said purified laccase,
characterized in that said host cell prepared in step a) is a Pichia pastoris yeast strain transformed with the pFD56 vector and in that the expression of the laccase is induced by adding methanol.

32. A method for preparing a laccase, comprising the steps of:
a) preparing host cells, as claimed in claim 30;
b) culturing host cells prepared in step a);
c) recovering the culture medium and removing said host cells in the case of secreted proteins, or lysing the host cells;
d) treating the medium or lysate obtained in step c) by hydrophobic interaction chromatography;
e) recovering said purified laccase,
characterized in that said host cell prepared in step a) is a Pichia pastoris yeast strain transformed with the pFD56 vector and in that the expression of the laccase is induced by adding methanol.

33. A laccase-containing electrode comprising a conductive material covered with a deposit comprising at least one laccase as claimed in claim 1.

34. A laccase-containing electrode comprising a conductive material covered with a deposit comprising at least one laccase as claimed in claim 2.

35. An oxygen, phenolic-compound or organic-amine biosensor, characterized in that it consists of an electrode as claimed in claim 33.

36. An oxygen, phenolic-compound or organic-amine biosensor, characterized in that it consists of an electrode as claimed in claim 34.

37. An oxygen sensor, characterized in that it consists of an electrode as claimed in claim 33.

38. An oxygen sensor, characterized in that it consists of an electrode as claimed in claim 34.

39. An enzymatic biofuel cell comprising an anode on which is immobilized an enzyme which catalyzes an oxidation reaction and an electrode as claimed in claim 33 as cathode.

40. An enzymatic biofuel cell comprising an anode on which is immobilized an enzyme which catalyzes an oxidation reaction and an electrode as claimed in claim 33 as cathode.


SEQUENCE LISTING (6 sequences)

SEQ ID NO: 1 - protein, 565 aa, Podospora anserina
VARKDWESPTYSWLYQFPLPIPPVKTPKLTVTNLVTGNPIHYYEVYINRFTQQVYPNKGPATLVGYDGISPGPTFIVERGHEAVVRFVNNASIENSVHLHGSYSRAPWDGWAEDVTMPGEFKDYYYPNQQAARFLWYHDHAFMHTAENAYFGQAGAYIIHDPAEDALNLPSGYGVHDIPLVLSSKQYNNNGSLFTTNGETDSLFGDVIHVNGQPWPYFNVEPRKYRLRFLDAAVSRTFKLYFQRQTGSSAKIPFQVIATDAGLMASPATTNDLYISMGERYEVVFDFSPFAGQNITLRNTDDVGQDDDYLHTNKVMRFIVGNTPVTDTSSIPSTLATVDWPTSDGTGVDRHFKFDRSNGEWQINGVVFADVNNRVLANVPRGKVEIWELENGGGGWSHPIHIHLVDFKVLWRSNDDGRPVYNYEAQGLKDVVWLAPNEIVRVEAHYAPWDGVYMFHCHNLIHEDHDMMAAFNVTALTDLGYNETAFRDPMEARWRAEPVTAAKFTTAAITEKIQFMAGLQPYNNVEEVLEVLDEYWATHSKRDAQDPAPKTRRMRIEGGKVKEVR

SEQ ID NO: 2 - DNA, 1788 bp, Podospora anserina
atgttgtccatcctcacttccgctcttctcgccgtctcggcctcgcctgttgtcttgggtcgggctacgcctgagcaaaagcacaagctggtcgccagaaaggattgggagagtcctacctattcgtggctttaccaatttcctctgcctattcctccggtcaagacgcccaaactgaccgttaccaacctggtgactggcaaccccattcactattacgaggtctacatcaaccggttcacccaacaagtgtatccaaacaagggtcccgcaactcttgttggttacgacggcatctctcccggcccgacctttatcgtggagagaggtcatgaggctgttgtgcgcttcgtcaacaatgccagcatcgaaaactcggtccatctccacgggtcatactctcgcgctccttgggatggttgggcagaggatgtgaccatgccaggcgagttcaaggactactactatcccaaccaacaggcggcccgtttcctgtggtaccacgatcacgcattcatgcatactgctgagaatgcatattttggccaagctggggcctacattattcatgatcctgctgaagacgccttgaaccttccctcaggttacggtgttcatgatattcctctggtgctctcttccaagcagtacaacaacaatggcagtctgttcaccaccaatggtgagacggactcgctctttggcgatgttatccacgtcaacggccagccctggccctacttcaacgttgagcctcgcaagtaccgcctccgcttcctcgacgctgccgtctcacgcaccttcaagctctacttccagcgtcaaactggcagctcagccaagattcctttccaagtcattgccacagatgccggcttgatggcctcaccagccaccacaaacgacctttacatcagcatgggagagcggtacgaggttgtctttgacttttcgccctttgctggccaaaacatcaccttgcgcaacacggacgacgtgggccaggacgacgattacctccacaccaacaaggtcatgagattcatcgttggcaacaccccagtgaccgacaccagctccattcccagcactctggctactgtcgactggccgacctctgatggaaccggtgtggacagacactttaagtttgaccgctccaatggcgagtggcagatcaacggagtcgtctttgccgatgtcaacaaccgtgtgcttgccaatgtcccccgcggcaaggttgaaatttgggagctcgagaacggcggcggcggatggagccacccaatccacattcacctggtcgacttcaaagtcttgtggcgctcgaatgacgatggcagaccggtctacaactacgaggcccagggtttgaaggatgttgtctggcttgcgcccaacgagattgtcagagtggaggcccactacgccccatgggacggagtctacatgttccactgccacaacctgattcacgaggatcacgacatgatggctgccttcaacgtcactgctctgactgacttgggatacaacgagaccgcgttccgtgaccccatggaggccagatggagagccgagccggtcacggctgccaagttcaccacggctgccatcaccgaaaagattcagttcatggccggactgcagccctacaacaatgtcgaggaagtcttggaggttctcgacgagtactgggccacgcacagcaagcgtgacgcccaagaccctgctcccaagaccagaagaatgagaattgagggcgggaaggtcaaagaggtccggtga

SEQ ID NO: 3 - protein, 566 aa, Podospora anserina
SVARKDWESPTYSWLYQFPLPIPPVKTPKLTVTNLVTGNPIHYYEVYINRFTQQVYPNKGPATLVGYDGISPGPTFIVERGHEAVVRFVNNASIENSVHLHGSYSRAPWDGWAEDVTMPGEFKDYYYPNQQAARFLWYHDHAFMHTAENAYFGQAGAYIIHDPAEDALNLPSGYGVHDIPLVLSSKQYNNNGSLFTTNGETDSLFGDVIHVNGQPWPYFNVEPRKYRLRFLDAAVSRTFKLYFQRQTGSSAKIPFQVIATDAGLMASPATTNDLYISMGERYEVVFDFSPFAGQNITLRNTDDVGQDDDYLHTNKVMRFIVGNTPVTDTSSIPSTLATVDWPTSDGTGVDRHFKFDRSNGEWQINGVVFADVNNRVLANVPRGKVEIWELENGGGGWSHPIHIHLVDFKVLWRSNDDGRPVYNYEAQGLKDVVWLAPNEIVRVEAHYAPWDGVYMFHCHNLIHEDHDMMAAFNVTALTDLGYNETAFRDPMEARWRAEPVTAAKFTTAAITEKIQFMAGLQPYNNVEEVLEVLDEYWATHSKRDAQDPAPKTRRMRIEGGKVKEVR

SEQ ID NO: 4 - DNA, 1695 bp, Podospora anserina
gtcgccagaaaggattgggagagtcctacctattcgtggctttaccaatttcctctgcctattcctccggtcaagacgcccaaactgaccgttaccaacctggtgactggcaaccccattcactattacgaggtctacatcaaccggttcacccaacaagtgtatccaaacaagggtcccgcaactcttgttggttacgacggcatctctcccggcccgacctttatcgtggagagaggtcatgaggctgttgtgcgcttcgtcaacaatgccagcatcgaaaactcggtccatctccacgggtcatactctcgcgctccttgggatggttgggcagaggatgtgaccatgccaggcgagttcaaggactactactatcccaaccaacaggcggcccgtttcctgtggtaccacgatcacgcattcatgcatactgctgagaatgcatattttggccaagctggggcctacattattcatgatcctgctgaagacgccttgaaccttccctcaggttacggtgttcatgatattcctctggtgctctcttccaagcagtacaacaacaatggcagtctgttcaccaccaatggtgagacggactcgctctttggcgatgttatccacgtcaacggccagccctggccctacttcaacgttgagcctcgcaagtaccgcctccgcttcctcgacgctgccgtctcacgcaccttcaagctctacttccagcgtcaaactggcagctcagccaagattcctttccaagtcattgccacagatgccggcttgatggcctcaccagccaccacaaacgacctttacatcagcatgggagagcggtacgaggttgtctttgacttttcgccctttgctggccaaaacatcaccttgcgcaacacggacgacgtgggccaggacgacgattacctccacaccaacaaggtcatgagattcatcgttggcaacaccccagtgaccgacaccagctccattcccagcactctggctactgtcgactggccgacctctgatggaaccggtgtggacagacactttaagtttgaccgctccaatggcgagtggcagatcaacggagtcgtctttgccgatgtcaacaaccgtgtgcttgccaatgtcccccgcggcaaggttgaaatttgggagctcgagaacggcggcggcggatggagccacccaatccacattcacctggtcgacttcaaagtcttgtggcgctcgaatgacgatggcagaccggtctacaactacgaggcccagggtttgaaggatgttgtctggcttgcgcccaacgagattgtcagagtggaggcccactacgccccatgggacggagtctacatgttccactgccacaacctgattcacgaggatcacgacatgatggctgccttcaacgtcactgctctgactgacttgggatacaacgagaccgcgttccgtgaccccatggaggccagatggagagccgagccggtcacggctgccaagttcaccacggctgccatcaccgaaaagattcagttcatggccggactgcagccctacaacaatgtcgaggaagtcttggaggttctcgacgagtactgggccacgcacagcaagcgtgacgcccaagaccctgctcccaagaccagaagaatgagaattgagggcgggaaggtcaaagaggtccgg

SEQ ID NO: 5 - protein, 595 aa, Podospora anserina
MLSILTSALLAVSASPVVLGRATPEQKHKLVARKDWESPTYSWLYQFPLPIPPVKTPKLTVTNLVTGNPIHYYEVYINRFTQQVYPNKGPATLVGYDGISPGPTFIVERGHEAVVRFVNNASIENSVHLHGSYSRAPWDGWAEDVTMPGEFKDYYYPNQQAARFLWYHDHAFMHTAENAYFGQAGAYIIHDPAEDALNLPSGYGVHDIPLVLSSKQYNNNGSLFTTNGETDSLFGDVIHVNGQPWPYFNVEPRKYRLRFLDAAVSRTFKLYFQRQTGSSAKIPFQVIATDAGLMASPATTNDLYISMGERYEVVFDFSPFAGQNITLRNTDDVGQDDDYLHTNKVMRFIVGNTPVTDTSSIPSTLATVDWPTSDGTGVDRHFKFDRSNGEWQINGVVFADVNNRVLANVPRGKVEIWELENGGGGWSHPIHIHLVDFKVLWRSNDDGRPVYNYEAQGLKDVVWLAPNEIVRVEAHYAPWDGVYMFHCHNLIHEDHDMMAAFNVTALTDLGYNETAFRDPMEARWRAEPVTAAKFTTAAITEKIQFMAGLQPYNNVEEVLEVLDEYWATHSKRDAQDPAPKTRRMRIEGGKVKEVR

SEQ ID NO: 6 - DNA, 5245 bp, Artificial Sequence
  Description of Artificial Sequence Synthetic polynucleotide
ggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatcagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctagcgtcgccagaaaggattgggagagtcctacctattcgtggctttaccaatttcctctgcctattcctccggtcaagacgcccaaactgaccgttaccaacctggtgactggcaaccccattcactattacgaggtctacatcaaccggttcacccaacaagtgtatccaaacaagggtcccgcaactcttgttggttacgacggcatctctcccggcccgacctttatcgtggagagaggtcatgaggctgttgtgcgcttcgtcaacaatgccagcatcgaaaactcggtccatctccacgggtcatactctcgcgctccttgggatggttgggcagaggatgtgaccatgccaggcgagttcaaggactactactatcccaaccaacaggcggcccgtttcctgtggtaccacgatcacgcattcatgcatactgctgagaatgcatattttggccaagctggggcctacattattcatgatcctgctgaagacgccttgaaccttccctcaggttacggtgttcatgatattcctctggtgctctcttccaagcagtacaacaacaatggcagtctgttcaccaccaatggtgagacggactcgctctttggcgatgttatccacgtcaacggccagccctggccctacttcaacgttgagcctcgcaagtaccgcctccgcttcctcgacgctgccgtctcacgcaccttcaagctctacttccagcgtcaaactggcagctcagccaagattcctttccaagtcattgccacagatgccggcttgatggcctcaccagccaccacaaacgacctttacatcagcatgggagagcggtacgaggttgtctttgacttttcgccctttgctggccaaaacatcaccttgcgcaacacggacgacgtgggccaggacgacgattacctccacaccaacaaggtcatgagattcatcgttggcaacaccccagtgaccgacaccagctccattcccagcactctggctactgtcgactggccgacctctgatggaaccggtgtggacagacactttaagtttgaccgctccaatggcgagtggcagatcaacggagtcgtctttgccgatgtcaacaaccgtgtgcttgccaatgtcccccgcggcaaggttgaaatttgggagctcgagaacggcggcggcggatggagccacccaatccacattcacctggtcgacttcaaagtcttgtggcgctcgaatgacgatggcagaccggtctacaactacgaggcccagggtttgaaggatgttgtctggcttgcgcccaacgagattgtcagagtggaggcccactacgccccatgggacggagtctacatgttccactgccacaacctgattcacgaggatcacgacatgatggctgccttcaacgtcactgctctgactgacttgggatacaacgagaccgcgttccgtgaccccatggaggccagatggagagccgagccggtcacggctgccaagttcaccacggctgccatcaccgaaaagattcagttcatggccggactgcagccctacaacaatgtcgaggaagtcttggaggttctcgacgagtactgggccacgcacagcaagcgtgacgcccaagaccctgctcccaagaccagaagaatgagaattgagggcgggaaggtcaaagaggtccggtgagc